(19) United States
(12) Patent Application Publication
(10) Pub. No.: US 2020/0182889 A1
(43) Pub. Date: Jun. 11, 2020

(54) CHEMILUMINESCENT BIOSENSOR FOR DETECTING COAGULATION FACTORS

(71) Applicant: Ji Hoon LEE, Gaithersburg, MD (US)

(72) Inventor: Ji Hoon LEE, Gaithersburg, MD (US)

(21) Appl. No.: 16/316,346

(22) PCT Filed: Jul. 17, 2017
(86) PCT No.: PCT/US2017/042404

§ 371 Date: Jan. 8, 2019

Related U.S. Application Data
(60) Provisional application No. 62/363,011, filed on Jul. 15, 2016.

(51) Int. Cl. G01N 33/86; G01N 21/76; C09K 11/07

(52) U.S. Cl. CPC G01N 33/86; G01N 21/76; C09K 2211/1018; G01N 2333/96444; G01N 2333/96463; C09K 11/07

(57) ABSTRACT

A chemiluminescent biosensor for detecting a coagulation factor in a blood sample including: a fluorogenic substrate for the coagulation factor, where the fluorogenic substrate includes a fluorescent dye; and a quencher conjugated with the fluorogenic substrate. The biosensor rapidly and accurately detects a coagulation factor in a blood sample including whole blood or plasma, thereby useful for minimizing or eliminating any reversal effect of anticoagulants.

This application claims the benefits of U.S. Provisional Application No. 62/363,011, filed Jul. 15, 2016, which is incorporated herein by reference in its entirety.

### TECHNICAL FIELD

The present disclosure relates to a chemiluminescent biosensor for detecting a coagulation factor in a blood sample within a very short period of time, a method of monitoring a coagulation factor, a method of quantifying a coagulation factor in a blood sample and a kit for quantifying a coagulation factor in a blood sample. The biosensor includes a fluorogenic substrate for the coagulation factor, wherein the fluorogenic substrate includes a fluorescent dye; and a quencher conjugated with the fluorogenic substrate.

### BACKGROUND ART

Normal coagulation, the process of forming a clot, is very important in an injury with bleeding because the process stops the bleeding so that the wound can heal. However, the blood should not clot while moving through the body because it can cause hypercoagulable states or thrombophilia. Blood clots in the veins or venous system, capable of travelling through the bloodstream, can cause deep vein thrombosis or a pulmonary embolus. In addition, blood clots in arteries can obstruct the flow of blood to major organs. Thus, arterial thrombosis can cause several serious conditions such as heart attack, stroke, severe leg pain, difficulty walking, and the loss of a limb.

In order to prevent bleeding, thrombosis, and stroke, various types of anticoagulants have been developed. In particular, anticoagulants are widely used as agents for the prevention and treatment of a myriad of cardiovascular conditions. Anticoagulants have been developed to control the activity (concentration) of coagulation factors (e.g., IIa, Xa) shown in FIG. 1A. This is because bleeding, thrombosis, and stroke can be prevented with the reduction of the activity of factors IIa or Xa using an appropriate anticoagulant. Recently, we have seen an increase in the usage of oral anticoagulants (DOACs) instead of conventional anticoagulant agents such as Warfarin, Phenprocoumon, Coumadin®, and heparin. Currently. DOACs inhibiting factor Xa (e.g., rivaroxaban, apixaban, edixaban) and IIa (e.g., dabigatran) are widely used.

Anticoagulants can prevent or treat acute or chronic thromboembolic diseases. However, the reversal effect of anticoagulant agents, such as excessive bleeding, may cause long-term debilitating diseases or be life-threatening. In general, the reversal effect of anticoagulants may take place immediately or in a few hours. The best method to accurately monitor the effect of anticoagulants may be the rapid quantification of a specific coagulation factor (e.g., IIa and Xa) which remains active after the intake of the anticoagulant by the patient. Unfortunately, analytical methods capable of directly quantifying coagulation factors in a few minutes are not yet available.

International normalized ratio (INR) which can be used for measuring coagulation time in in-vitro conditions, as an alternative method, is widely used to study the effect of factor IIa anticoagulants. However, it is difficult to predict the reversal effect of IIa anticoagulants using the INR because the value of INR is dependent on the disease of the patient. For example, the value of INR determined from a patient with prosthetic heart valves is lower than the expected INR target range.

Recently, a number of highly sensitive biosensors using two DNA aptamers have been developed for quantifying factor IIa. However, they cannot be applied to accurately and rapidly predict the reversal effect of factor IIa anticoagulants because DNA aptamer cannot rapidly bind to factor IIa in human samples (e.g., plasma, whole blood). In order to enhance the binding rate between DNA aptamer and factor IIa, a human sample was 100˜10,000-fold diluted with appropriate buffers. Additionally, multiple-time incubations and washings are necessary for quantifying factor IIa using biosensors. Thus, these biosensors cannot be used to rapidly monitor the reversal effect of anticoagulants.

INR and a partial thromboplastin time (aPTT) are not appropriate for the evaluation of factor Xa anticoagulants. However, sandwich enzyme immunoassay, using two monoclonal antibodies that binds to the factor Xa anticoagulants, can be used to study the efficiency of factor Xa anticoagulants. But, it is still difficult to rapidly predict the reversal effect of factor Xa anticoagulants using the time-consuming sandwich enzyme immunoassay.

Proteases, which act as an enzyme in the body, can recognize and hydrolyze specific endogenous peptides and proteins by binding their amino acid side chains. Specific endogenous peptides and proteins are substrates capable of reacting with a specific enzyme. Using the hydrolysis reaction, various types of biosensors with absorbance and fluorescence detection have been developed for the quantification and monitoring of a specific protease, a biomarker applied to early diagnose human diseases. FIG. 1B shows the basic concept for the reaction between a protease and substrate conjugated with a chromophore or fluorescent dye to measure absorbance or fluorescence. With the increase of protease concentration, the absorbance or fluorescence intensity is enhanced. Factors IIa and Xa are known as protease proteins. Thus, various substrates capable of reacting with factors IIa or Xa have been developed. Also, multiple biosensors with UV-visible absorbance or fluorescence detection have been developed for quantifying factor IIa or Xa. Unfortunately, these biosensors are not appropriate because the time necessary for quantifying IIa or Xa in human samples is too long to monitor the reversal effect of IIa or Xa coagulants in a few minutes.

It has been known that 1,1′-Oxalyldiimidazole chemiluminescence (ODI-CL), generated from the reaction mechanism shown in FIG. 1C, is 10-1000-fold more sensitive than absorbance and fluorescence detection. Also, the dynamic range of a biosensor with ODI-CL detection is much wider than those with absorbance or fluorescence detections. Luminophore shown in FIG. 1C is a fluorescent dye capable of receiving energy from high-energy intermediate to emit bright and rapid luminescence as shown in FIG. 1D.

However, a highly sensitive biosensor detecting/quantifying a coagulation factor (e.g., IIa, Xa) in a blood sample within a few minutes, so as to minimize or eliminate any adverse reversal effects, has yet to be developed.

### SUMMARY

According to one aspect of the present invention, a biosensor for detecting a coagulation factor in a blood sample is provided, which comprises: a fluorogenic substrate for the coagulation factor, wherein the fluorogenic substrate includes a fluorescent dye; and a quencher conjugated with the fluorogenic substrate. The coagulation factor may be coagulation factor IIa or Xa, and the blood sample is plasma or whole blood. The blood sample may be 1 to 1,000-fold diluted plasma or whole blood. The fluorescent dye may be at least one selected from the group consisting of 2-aminobenzoyl (Abz), N-methyl-anthraniloyl (N-Me-Abz), 5-(dimethylamino)naphthalene-1-sulfonyl (Dansyl), 5-[2-aminoethyl)amino]-naphthalene-1-sulfonic acid (EDANS), 7-dimethylaminocoumarin-4-acetate (DMACA), 7-amino-4-methylcoumarin (AMC), (7-methoxycoumarin-4-yl)acetyl (MCA), rhodamine, rhodamine 101, rhodamine 110 and resorufin. The fluorescent dye may emit light when: the fluorescent dye dissociates from the fluorogenic substrate by a hydrolysis reaction between the coagulation factor and the fluorogenic substrate, and when the fluorescent dye interacts with high-energy intermediate formed from 1,1′-oxalyldiimidazole chemiluminescence (ODI-CL) reagent. The 1,1′-oxalyldiimidazole chemiluminescence (ODI-CL) reagent may comprise an ODI and H₂O₂. The quencher is at least one selected from the group consisting of 2,4-Dinitrophenyl (DNP), N-(2,4-Dinitrophenyl)ethylenediamine (EDDnp), 4-Nitro-phenylalanine, 3-Nitro-tyrosine, para-Nitroaniline (pNa), 4-(4-Dimethylaminophenylazo)benzoyl (DABCYL) and 7-Nitro-benzo[2,1,3]oxadiazol-4-yl (NBD).

In accordance with another aspect of the present invention, a method of monitoring a coagulation factor in a blood sample, comprises: mixing and reacting the biosensor with a blood sample including a coagulation factor in a buffer; adding a 1,1′-oxalyldiimidazole chemiluminescence (ODI-CL) reagent to the reacted mixture; and measuring CL intensity. The reaction time between the blood sample and the fluorogenic substrate in the biosensor at room temperature (21±2° C.) or 37° C. may be 10 second to 120 minutes. The measuring CL intensity may be performed for 1 to 10 seconds after adding the ODI-CL reagent. The coagulation factor may be coagulation factor IIa or Xa, and the blood sample may be plasma or whole blood. The buffer may be selected from the groups consisting of PBST, PBS, TBST and TBS.

In yet another aspect of the present invention, a method of quantifying a coagulation factor in a blood sample, comprises: mixing and reacting the biosensor with a blood sample including a coagulation factor in a buffer; adding 1,1′-oxalyldiimidazole chemiluminescence (ODI-CL) reagent to the reacted mixture; measuring CL intensity; and comparing the CL intensity with a standard intensity.

In yet another aspect of the present invention, a kit for quantifying a coagulation factor in a blood sample, comprises: the biosensor; and a container. The kit may further comprise a buffer; and 1,1′-oxalyldiimidazole chemiluminescence (ODI-CL) reagent.

These and other aspects will be appreciated by one of ordinary skill in the art upon reading and understanding the following specification.

### BRIEF DESCRIPTION OF THE DRAWINGS

FIG. 1A shows the role of Xa and IIa in the blood coagulation cascade.

FIG. 1B is a diagram for the hydrolysis reaction between protease and substrate conjugated with chromophore or fluorescent dye.

FIG. 1C shows a reaction mechanism of 1,1′ -Oxalyldiimidazole chemiluminescence (ODI-CL), where L is luminophore under the ground state and L* is luminophore under the excited state.

FIG. 1D is a graph showing a rapid ODI-CL spectrum.

FIG. 2A is a graph showing relative CL intensities in the absence and presence of the coagulation factor IIa (5 nM) or Xa (5 nM).

FIG. 2B depicts chemiluminescent resonance energy transfer (CRET) in the absence of biomarker such as factors IIa and Xa in plasma.

FIG. 2C depicts ODI-CL reaction in the presence of fluorogenic substrate and protease enzyme.

FIG. 2D shows hydrolysis reaction between the fluorogenic substrate and coagulation factor IIa or Xa.

FIG. 2E shows ODI-CL reaction in the presence of a fluorescent dye (e.g., AMC) formed from the hydrolysis reaction between fluorogenic substrate and coagulation factors (e.g., IIa, Xa)

FIG. 3A is a graph showing the effect of plasma in the presence of coagulation factor IIa using a specific substrate conjugated AMC in PBS.

FIG. 3B is a graph showing the effect of plasma in the presence of coagulation factor Xa using a specific substrate conjugated AMC in PBS.

FIG. 3C is a graph showing the selection of buffer for the quantification of coagulation factor IIa (6.8 nM) in 10% human plasma.

FIG. 3D is a graph showing the selection of buffer for the quantification of coagulation factor Xa (10 nM) in 10% human plasma.

FIG. 4A is a graph showing the calibration curves for the quantification of coagulation factor IIa with the rapid biosensor with ODI-CL detection.

FIG. 4B is a graph showing the calibration curves for the quantification of coagulation factor Xa with the rapid biosensor with ODI-CL detection.

FIG. 4C is a graph showing the correlations (N=10) between ODI-CL and fluorescence detection for the quantification of coagulation factor IIa in human plasma. (The error range of each value were 4-7%).

FIG. 4D is a graph showing the correlations (N=10) between ODI-CL and fluorescence detection for the quantification of coagulation factor Xa in human plasma. (The error range of each value were 4-7%).

FIG. 5A is a graph showing the effect of incubation time in the absence and presence of coagulation factor IIa in whole blood.

FIG. 5B is a graph showing the effect of incubation time in the absence and presence of coagulation factor Xa in whole blood.

FIG. 5C is a graph showing the enhancement of relative CL intensity with the extension of reaction (incubation) time between IIa fluorogenic substrate and coagulation factor IIa in whole blood (N=5).

FIG. 5D is a graph showing the enhancement of relative CL intensity with the extension of reaction (incubation) time between Xa fluorogenic substrate and coagulation factor Xa in whole blood (N=5).

FIG. 6A is a graph showing the calibration curves capable of rapidly quantifying trace levels of coagulation factor IIa in whole blood using the biosensor with ODI-CL detection.

FIG. 6B is a graph showing the calibration curves capable of rapidly quantifying trace levels of coagulation factor Xa in whole blood using the biosensor with ODI-CL detection.

FIG. 6C is a graph showing the selectivity and specificity of Xa fluorogenic substrate conjugated with AMC.

FIG. 6D is a graph showing the selectivity and specificity of IIa fluorogenic substrate conjugated with AMC.

FIG. 7 is a graph showing the effect of plasma in ODI-CL reaction in the presence of AMC (12.5 μM) as a luminophore (fluorescent dye). Each sample was prepared as a mixture (volume ratio=1:1) of AMC in TBST and a certain % concentration of human plasma diluted with H₂O.

FIG. 8 is a graph showing the relative CL intensity of AMC (25 μM) in four different buffer solutions.

FIG. 9 is a graph showing CL_{3.4}/CL₀ over different reaction (incubation) time for the quantification of IIa (3.4 nM) in 10% human plasma.

FIG. 10 is a graph showing the dilution effect for the quantification of Xa in human whole blood. Reaction time of the diluted whole blood and fluorogenic substrate is 2, 4, and 10 min.

FIG. 11 is a graph showing the effect of components in whole blood in ODI-CL reaction in the presence of AMC (25 μM) as a luminophore (fluorescent dye).

FIG. 12A is a graph showing the specificity and selectivity of IIa fluorogenic substrate applied to develop the biosensor with ODI-CL detection. Concentration: [IIa]=28.6 ng/ml, [Glucose]=10⁶ ng/ml, [Hemoglobin]=10⁶ ng/ml, [HSA]=10⁶ ng/ml, [IgG]=10⁶ ng/ml.

FIG. 12B is a graph showing the specificity and selectivity of Xa fluorogenic substrate applied to develop the biosensor with ODI-CL detection. Concentration: [Xa]=10.8 ng/ml, [Glucose]=10⁶ ng/ml, [Hemoglobin]=10⁶ ng/ml, [HSA]=10⁶ ng/ml, [IgG]=10⁶ ng/ml.

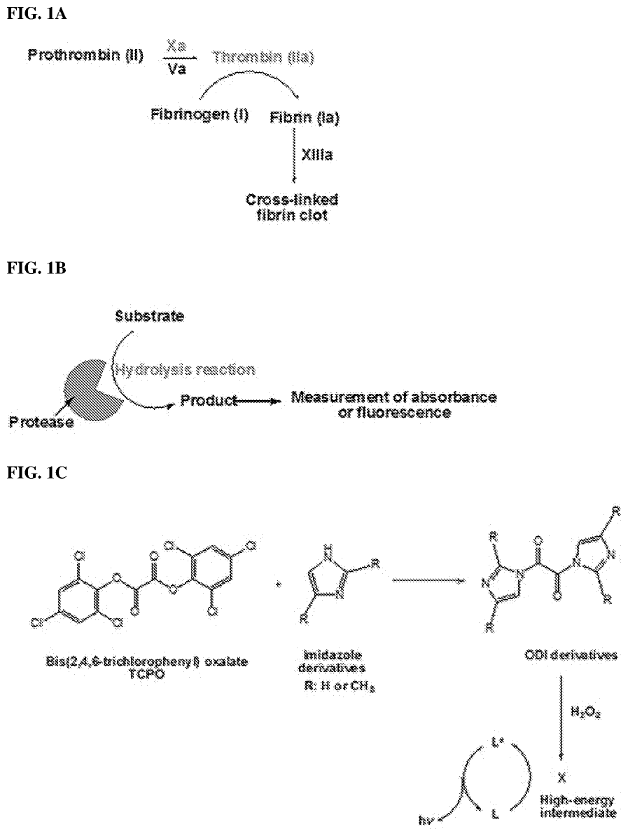

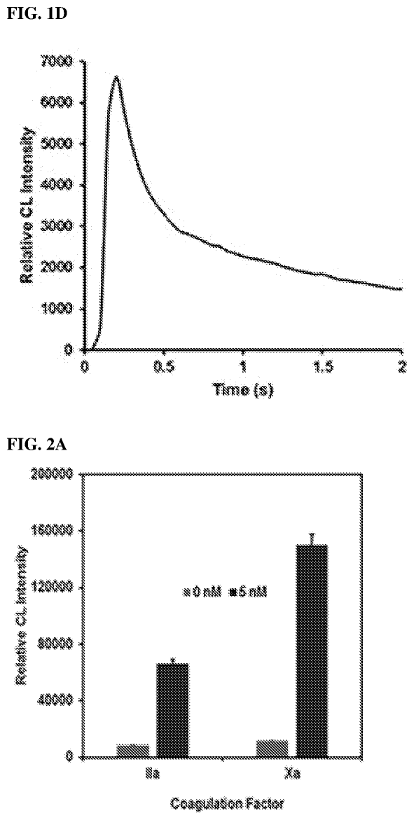

### DETAILED DESCRIPTION

According to an embodiment of the present invention, a biosensor is provided for detecting a coagulation factor in a blood sample, the biosensor comprises: a fluorogenic substrate for the coagulation factor, wherein the fluorogenic substrate includes a fluorescent dye; and a quencher conjugated with the fluorogenic substrate. The fluorescent dye emits light when the fluorescent dye is dissociated from the fluorogenic substrate by a hydrolysis reaction between the coagulation factor and the fluorogenic substrate, and when the fluorescent dye interacts with high-energy intermediate formed from 1,1′-oxalyldiimidazole chemiluminescence (ODI-CL) reagent. The 1,1′-oxalyldiimidazole chemiluminescence (ODI-CL) reagent may comprise an ODI and H₂O₂.

The fluorescent dye used in the fluorogenic substrate may be at least one of 2-aminobenzoyl (Abz), N-methyl-anthraniloyl (N-Me-Abz), 5-(dimethylamino)naphthalene-1-sulfonyl (Dansyl), 5-[(2-aminoethyeamino]-naphthalene-1-sulfonic acid (EDANS), 7-dimethylaminocoumarin-4-acetate (DMACA), 7-amino-4-methylcoumarin (AMC), (7-methoxycoumarin-4-yl)acetyl (MCA), rhodamine, rhodamine 101, rhodamine 110 and resorufin. In this specification, AMC is used as an example, but other fluorescent dye can be used alone or in combination with each other.

The quencher used in the biosensor may be at least one of 2,4-Dinitrophenyl (DNP), N-(2,4-Dinitrophenyl)ethylenediamine (EDDnp), 4-Nitro-phenylalanine, 3-Nitro-tyrosine, para-Nitroaniline (pNa), 4-(4-Dimethylaminophenylazo)benzoyl (DABCYL) and 7-Nitro-benzo[2,1,3]oxadiazol-4-yl (NBD).

The coagulation factor of the present invention can be any type of coagulation factor that is involved in the blood coagulation cascade. Among the various coagulation factors, factor IIa (thrombin) and factor Xa are preferable.

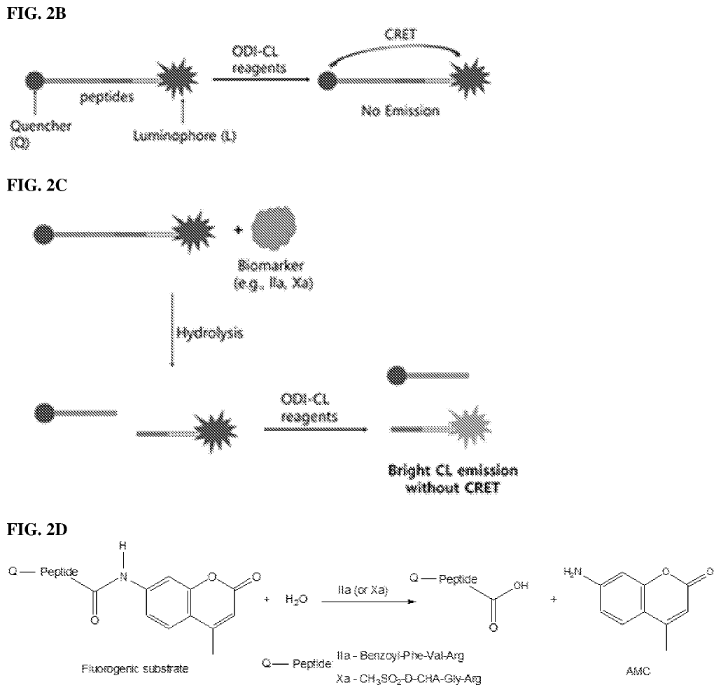

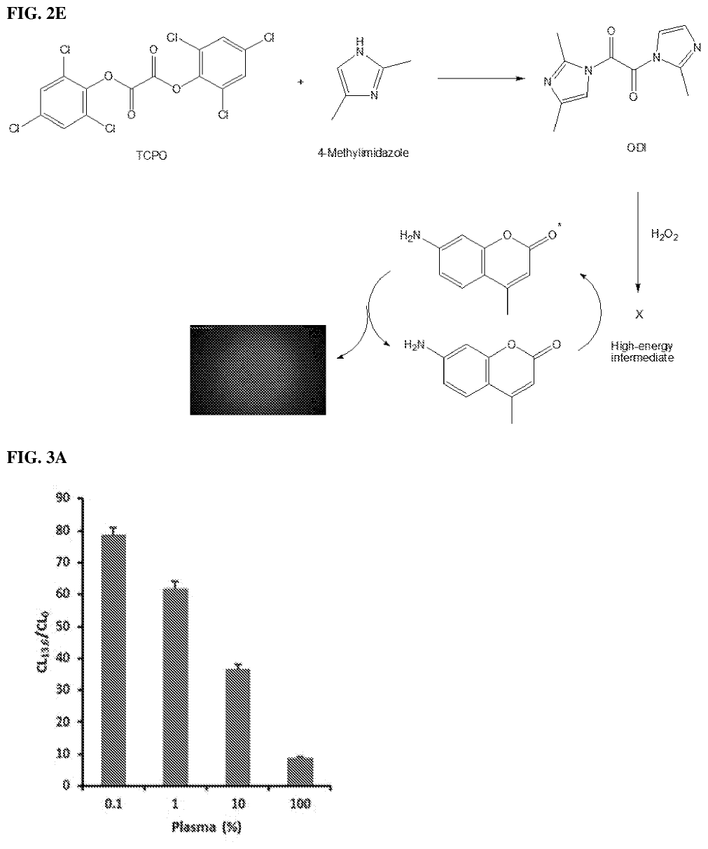

As shown in FIG. 2A, a fluorescent dye (e.g., AMC) in a fluorogenic substrate for the coagulation factor IIa (or Xa) does not emit light in an ODI-CL detection system in the absence of the coagulation factor IIa (or Xa). This is because the fluorescent dye (AMC; luminophore (L)) is excited by the high-energy intermediate formed from the reaction between the ODI and H₂O₂ transfer energy to the quencher (Q) conjugated with the fluorogenic substrate due to the chemiluminescent resonance energy transfer (CRET) as shown in FIG. 2B. FIG. 2A shows that relative CL intensity in the presence of the coagulation factor (5 nM) is much higher than that in the absence of the coagulation factor. The results can be illustrated by the reaction scheme shown in FIG. 2C. The fluorogenic substrate was separated by the hydrolysis reaction of the fluorogenic substrate and the coagulation factor. Thus, the fluorescent dye (luminophore) not bound with the quencher can emit light in the ODI-CL reaction. FIG. 2D shows that a fluorescent dye (AMC) is separated as a result of the hydrolysis reaction between the coagulation factor IIa (or Xa) and a specific fluorogenic substrate for the coagulation factor. In the example shown in FIG. 2E, AMC excited by the high-energy intermediate (X) formed from ODI-CL reaction can emit (blue) light.

An exemplary structure of a IIa specific fluorogenic substrate and an Xa specific fluorogenic substrate are shown TABLE 1 below.

TABLE 1

| Fluorogenic substrate | IIa | Xa |
|---|---|---|
| Formula | Benzoyl-Phe-Val-Arg- | CH₃SO₂-D-CHA-Gly-Arg- |
| | AMC, HCl | AMC, AcOH |
| Physical Form | Lyophilized | Lyophilized |
| Fluorescent dye Chemical Structure | 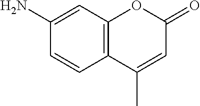 | 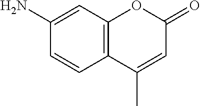 |
| Mw (g mol⁻¹) | 681.78 | 679.8 |
| Ex/Em Wavelength (nm) | 342/440 | 342/440 |
| Kₘ (μM) | 3.90 | 220 |
| k_{cat} (s⁻¹) | 42.7 | 162.0 |
| k_{cat}/Kₘ (s⁻¹ μM⁻¹) | 10.9 | 0.74 |

In the present invention, the blood sample can be either plasma or whole blood. The blood sample can be used as is, or 1 to 1,000-fold diluted. The effect of using plasma in ODI-CL reactions using AMC (12.5 μM) as a fluorescent dye is shown in FIG. 7. As shown, the relative CL intensity of AMC in 0.1˜10% plasma (10 to 1000-fold dilution) was constant within a statistically acceptable error range (<5%). The relative CL intensity in 100% plasma was lower than those in 0.1˜10% plasma because some components in human plasma may act as an inhibitor or quencher in ODI-CL reactions.

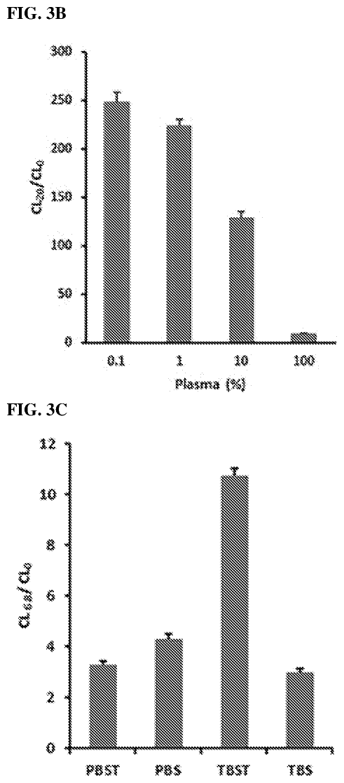

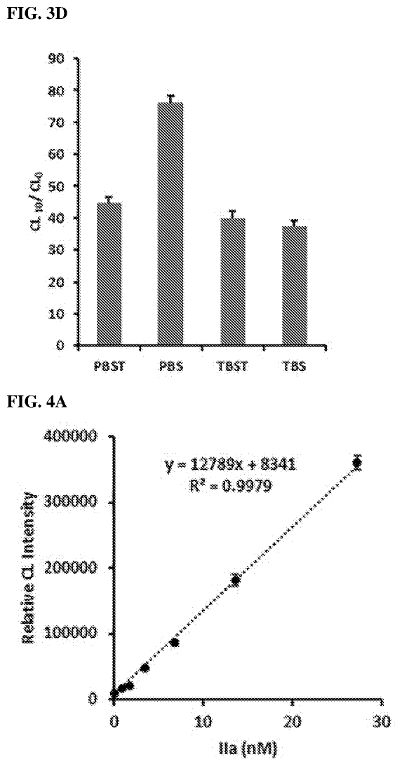

FIGS. 3A and 3B show the sensitivity of ODI-CL emitted in the biosensor depending on the concentration of the plasma. The signal/background ratio (CL_{IIa}/CL₀ or CL_{Xa}/CL₀) was enhanced when the composition of human plasma was diluted. Thus, the biosensor using 10 to 1000-fold diluted human plasma can be more sensitive than that using 100% human plasma. Additionally, the signal/background ratio in 10% human plasma was about 50% lower than that in 0.1% human plasma. These results indicate that the limit of detection (LOD=3σ) for the biosensor operated with 10-fold diluted human plasma may be as low as or slightly higher than that for the biosensors generated with 1,000-fold diluted human plasma. σ is the standard deviation of background measured in the absence of the coagulation factor IIa or Xa.

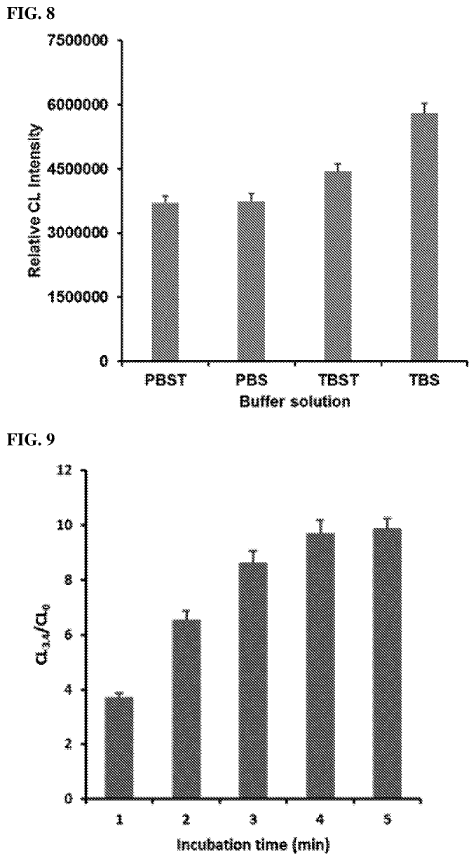

In the present invention, peptides specific to the coagulation factor included in the biosensor may react with a coagulation factor in a buffer. The buffer can be any one of Phosphate buffered saline with Tween-20 (PBST), Phosphate buffered saline (PBS), Tris buffered saline with Tween-20 (TBST) and Tris buffered saline (TBS). As shown in FIG. 8, the light emitted from AMC (25 μM) in TBS is brighter than those in other buffer solutions. The results imply that TBS is the best buffer solution of ODI-CL biosensor capable of rapidly quantifying trace levels of AMC formed from the hydrolysis reaction between the coagulation factor IIa (or Xa) and a specific substrate conjugated with AMC shown in FIG. 2D. However, FIGS. 3C and 3D indicate that the best buffer for the hydrolysis reaction between the coagulation factor IIa and the substrate conjugated with AMC is TBST, whereas PBS is the best buffer for the hydrolysis reaction between the coagulation factor Xa and the substrate. These results indicate that the yield of AMC formed from the hydrolysis reaction between the coagulation factor and a specific substrate conjugated with AMC is dependent on the type of buffer solution. For example, FIG. 3C shows that the relative CL intensity measured after the 2-min hydrolysis reaction in TBST is the strongest. Thus, the results shown in FIGS. 8 and 3C indicate that the concentration of AMC formed from the 2-min hydrolysis reaction in TBST is higher than those in the other buffer solutions. In other words, the hydrolysis reaction in TBST is faster than those in the other buffer solutions. As another example, FIG. 3D shows that the best buffer solution for the quantification of the coagulation factor Xa using the biosensor with ODI-CL detection is PBS because the concentration of AMC formed after the 2-min hydrolysis reaction in PBS is higher than those in PBST, TBS, and TBST. Based on the results, TBST may be preferable for monitoring/quantifying IIa in a human sample while PBS may be preferable for monitoring/quantifying Xa in a human sample.

In the present invention, the reaction (hydrolysis) time between the blood sample and the fluorogenic substrate in the biosensor at room temperature (21±2° C.) or 37° C. may be controlled in the range of approximately 10 seconds to 120 minutes. Preferably, the reaction (hydrolysis) time may be controlled to be 1-30 minutes, and most preferably, 1-4 minutes. FIG. 9 shows that the sensitivity of the biosensor with ODI-CL detection is dependent on the incubation time necessary for the hydrolysis reaction between the coagulation factor and the substrate conjugated with AMC. With an increase in the hydrolysis reaction (incubation) time, CL_{3.4}/CL₀ was enhanced. CL_{3.4}/CL₀ calculated with relative CL intensities measured after a 4-minute incubation was similar to that after a 5-minute incubation. Thus, it is possible to have the reaction (hydrolysis) time for quantifying coagulation factor IIa in 10% human plasma as 4 minutes using the biosensor with ODI-CL detection and the substrate conjugated with AMC in TBST. Additionally, in accordance with the exemplary embodiments of the present invention in FIG. 9, a 1-minute incubation time is also possible. This is because 3.4 nM detected after a 1-minute incubation (hydrolysis) is lower than the normal range (10˜15 nM) in 10% human plasma even though it is expected that the sensitivity of biosensor operated with a 1-minute incubation may not be as good as that generated after 4 minutes incubation. Thus, the biosensor with ODI-CL detection is possible for the rapid quantification of the coagulation factor IIa with a short incubation (1˜4 min) of the mixture of 10% human plasma and substrate conjugated with AMC in TBST.

The reaction (hydrolysis) time is also applicable to the biosensor capable of sensing the coagulation factor Xa in 10% human plasma in PBS. The following table shows a normalized intensity of ODI-CL and fluorescence (conventional) for quantifying factor Xa in 10% human plasma.

TABLE 2

| | Normalized | Normalized |
|---|---|---|
| Xₐ | ODI-CL Intensity | Fluorescence Intensity |
| (nM) | (2-min incubation) | (30-min incubation) |
| 0 | 2.51 | 4.22 |
| 0.02 | 3.46 | 4.92 |
| 0.05 | 3.94 | 4.08 |
| 0.11 | 4.42 | 4.22 |
| 0.27 | 5.26 | 5.63 |
| 0.62 | 6.57 | 6.61 |
| 1.45 | 8.48 | 9.14 |
| 3.37 | 11.11 | 13.50 |
| 7.87 | 17.68 | 20.68 |
| 18.37 | 27.12 | 39.24 |
| 42.86 | 48.51 | 63.01 |
| 100.00 | 100.00 | 100.00 |

*The error range of each value measured with ODI-CL or fluorescence detection was 3~7%.
**The excitation and emission wavelengths for the fluorescence measurement were 342 and 440 nm.

As shown in TABLE 2, the biosensor with ODI-CL detection is much more sensitive than a conventional sensor with fluorescence detection. ODI-CL was able to detect 0.02 nM Xₐ with only a 2-min incubation period under ambient conditions, whereas the fluorescence detection could not sufficiently sense 0.11 nM Xₐ even with the 30-min incubation due to the high background generated while operating light source. The sensitivity of the biosensor with the fluorescence detection, a conventional method, was used to compare with the biosensor with ODI-CL detection. (https://www.mybiosource.com/prods/Assay-Kit/Factor-Xa/datasheet.php?products_id.84634).

According to another embodiment of the present invention, a method for monitoring/quantifying a coagulation factor in a blood sample by using a biosensor as described above. The method includes mixing and reacting the biosensor with a blood sample including a coagulation factor in a buffer; adding a 1,1′-oxalyldiimidazole chemiluminescence (ODI-CL) reagent to the reacted mixture; and measuring CL intensity. The reaction (hydrolysis) time between the blood sample and the fluorogenic substrate in the biosensor at room temperature (21±2° C.) or 37° C. may be 10 seconds to 120 minutes, and the measuring CL intensity may be performed for 1 to 10 seconds after adding the ODI-CL reagent.

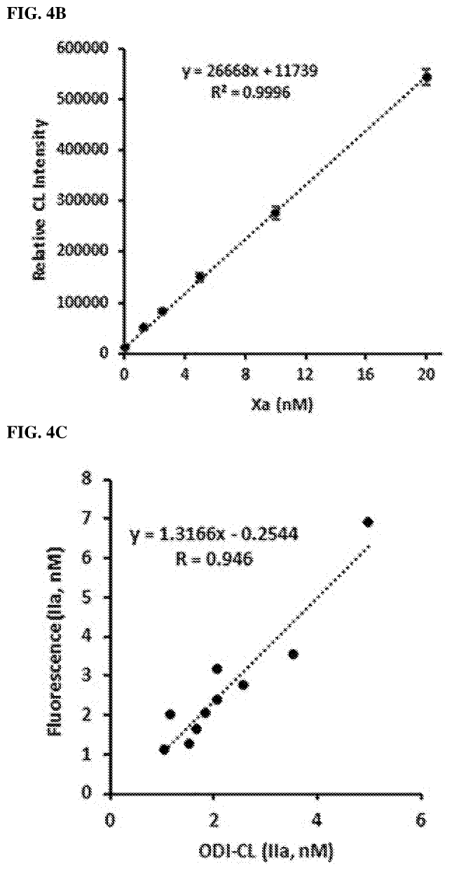

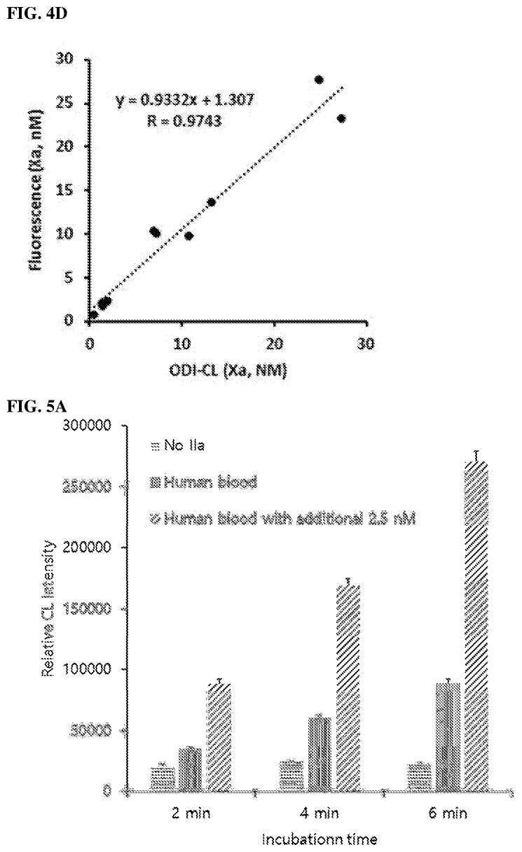

With a 2-min incubation of the coagulation factor IIa (and Xa) and a substrate conjugated with AMC, as shown in FIGS. 4A and 4B, the biosensor with ODI-CL detection can rapidly quantify trace levels of IIa and Xa with wide linear calibration curves. The dynamic range of linear calibration curves for quantifying factor IIa was as wide as 0.3 to 27.2 nM. The LOD of the biosensor capable of analyzing factor IIa was as low as 104 pM. Also, the dynamic range of linear calibration curves for the analysis of factor Xa was as wide as 0.25 to 20 nM. The LOD of the biosensor was as low as 44 pM. Using the biosensor with excellent linear calibration curves as in FIGS. 4A and 4B, the present invention achieves the accurate, cost-effective, and rapid quantification of a coagulation factor with just a 10-fold diluted plasma sample instead of 100˜10,000 times diluted plasma as in the case of previous biosensors. FIGS. 4C and 4D show a good correlation between a biosensor with ODI-CL detection and a conventional biosensor with fluorescence detection. These results indicate that a biosensor with ODI-CL detection with a 2-min incubation of the mixture (e.g., a specific fluorogenic substrate, factor IIa or Xa) may be a cost-effective, rapid, and easy-to-use diagnostic method for quantifying coagulation factors.

The following TABLE 3 shows that a biosensor with ODI-CL detection according to exemplary embodiments of the present invention can quantify coagulation factors IIa and Xa with good accuracy, precision, and recovery. Thus, a biosensor according to the present invention can quantify factors IIa and Xa in human plasma with a statistically acceptable reproducibility far more rapidly than conventional biosensors.

TABLE 3
(Accuracy, precision, and recovery for the all-in-one
Biosensor with ODI-CL detection for the quantization of
IIa and Xa in human plasma (N = 5))

| | Sample 1 | Sample 2 | Expected | Measured | Recovery |
|---|---|---|---|---|---|
| Factor | (nM) | (nM) | (nM) | (nM) | (%) |
| IIa | 6.8 | 27.6 | 17.2 | 16.52 ± 0.94 | 95.9 |
| | 8.5 | 14.0 | 11.25 | 11.94 ± 0.68 | 105.8 |
| Xa | 5.0 | 20.0 | 12.5 | 12.79 ± 0.43 | 101.6 |
| | 2.5 | 17.5 | 10.0 | 9.42 ± 0.52 | 94.2 |

Analyses of Factors IIa and Xa in Whole Blood

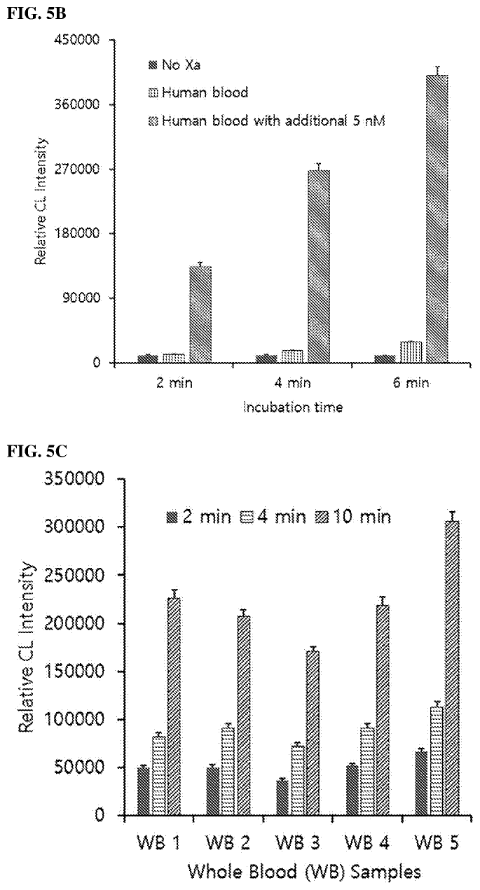

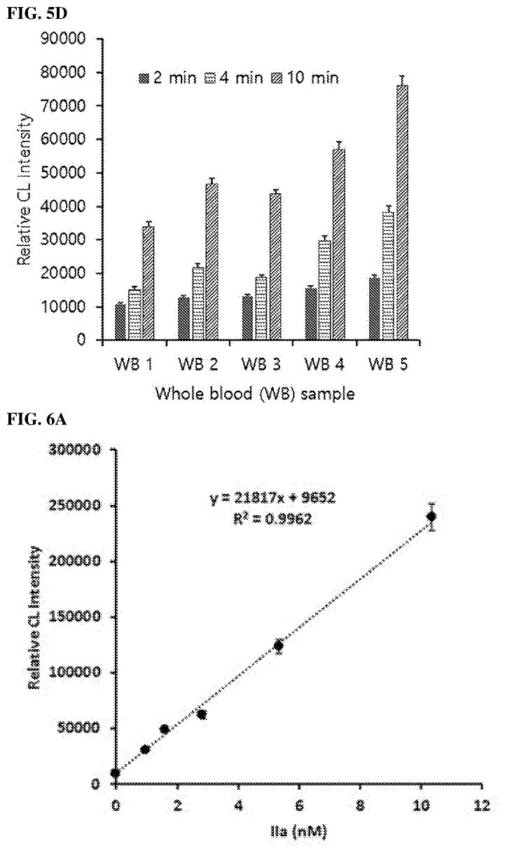

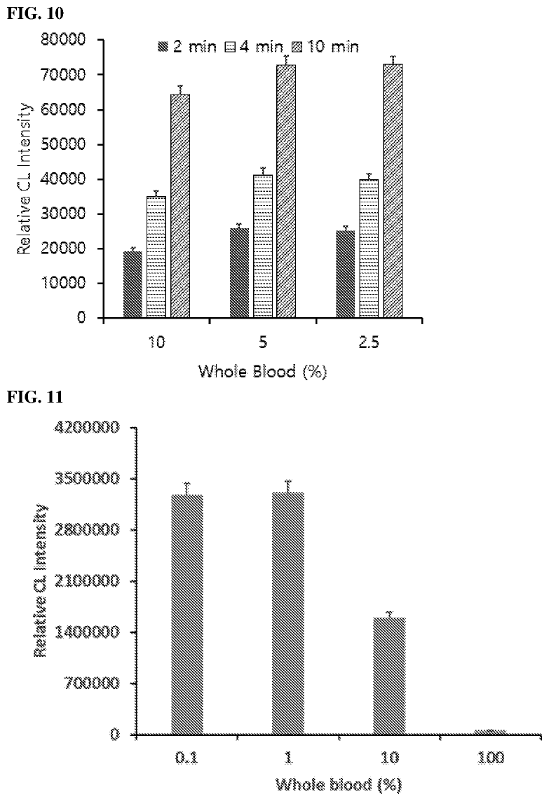

A biosensor according to exemplary embodiments of the present invention can be used with whole blood as the sample. FIG. 10 shows the application of the biosensor to a whole blood sample (where the whole blood sample was 10˜40 times diluted with deionized H₂O), which again indicates that a biosensor with ODI-CL detection can rapidly quantify trace levels of coagulation factors in 10-fold diluted whole blood similar to the quantification of factors IIa and Xa in 10-fold diluted plasma. FIGS. 5A and 5B also show that the factor IIa (or Xa) substrate conjugated with AMC is so stable in negative sample not containing IIa (or Xa) that the relative CL intensity (background) measured after three different incubation times of the mixture (e.g., negative sample and IIa (or Xa) substrate conjugated with AMC) was constant within the statistically acceptable error range (<5%). The strength of the light emitted in the patient sample (e.g., 10-fold diluted whole blood) containing trace levels of IIa and Xa proportionally increased with the extension of the incubation time. The relative CL intensity of the sample, spiked IIa (2.5 nM) or Xa (5 nM) in the patient sample, was higher than that of the pure patient sample as well being dependent on the incubation time.

As shown in FIGS. 5C and 5D, the relative CL intensity of a whole blood sample was different from those of the other four whole blood samples because IIa and Xa concentrations in each whole blood sample were different. However, the relative CL intensity of each whole blood sample was proportionally enhanced with the extension of the incubation time. Thus, FIGS. 5C and 5D indicate that a biosensor with ODI-CL detection can rapidly quantify trace levels of IIa (or Xa) in the patient sample (e.g., 10-fold diluted whole blood) with statistically acceptable precision and reproducibility.

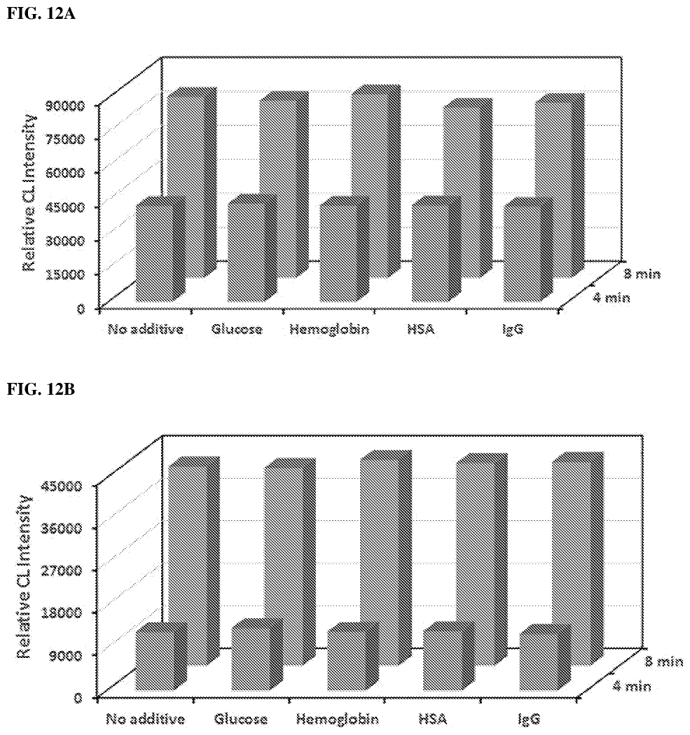

As shown in FIG. 11, the relative CL intensity of AMC in the 10% whole blood was about 50% lower than those in 0.1 and 1% whole blood, whereas the relative CL intensity of AMC in the 10% plasma was the same as those in 0.1 and 1% plasma (See FIG. 7). Also, the relative CL intensity of AMC in 100% whole blood was about 60-fold lower than those in 0.1 and 1% whole blood. The results shown in FIG. 11 indicate that the quantum efficiency of AMC emitted in ODI-CL reaction is decreased by some inhibitors existing in 10 and 100% whole blood samples. Based on the results shown in FIGS. 7 and 11, it is expected that human whole blood may contain some components capable of acting as a strong inhibitor in ODI-CL reaction. In order to overcome the disadvantages associated with using a whole blood sample in ODI-CL reactions, a 4-min incubation of the mixture (e.g., IIa (or Xa) substrate conjugated with AMC and 10-fold diluted whole blood sample) was selected for the development of a highly sensitive biosensor with ODI-CL detection capable of rapidly diagnosing and preventing bleeding, thrombosis, and stroke. Thus, preferably, the reaction (hydrolysis) time for the quantification of IIa and Xa in whole blood may be set 2 times longer than that in plasma.

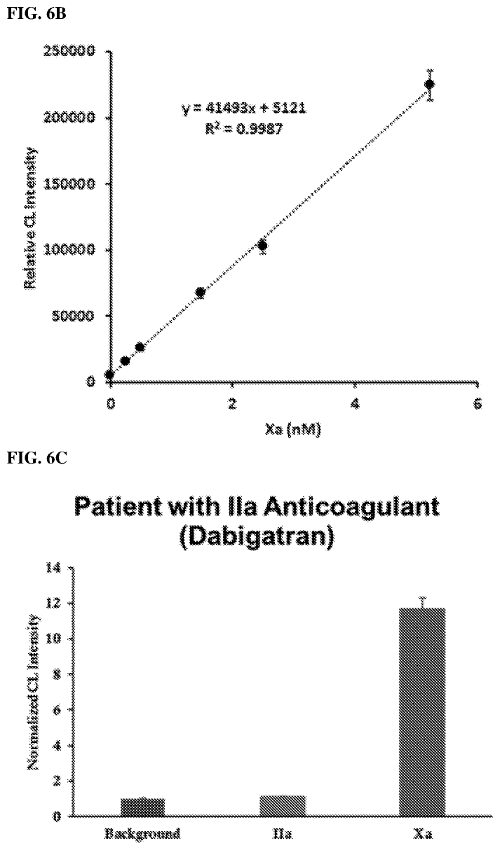

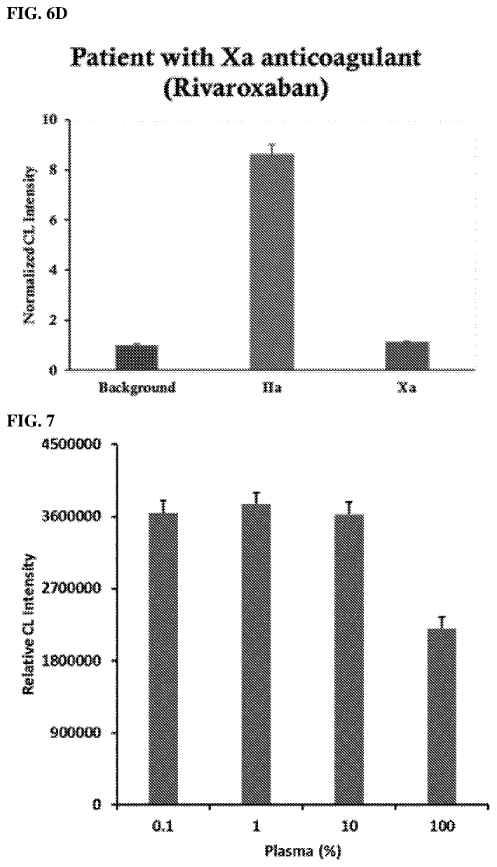

The linear calibration curves of FIGS. 6A and 6B indicate that a biosensor with ODI-CL detection, and with the 4-min incubation of the mixture, can rapidly quantify IIa and Xa in patient whole blood. LODs of the biosensor capable of quantifying IIa and Xa were as low as 66 and 18 pM in whole blood. LODs of the biosensor in whole blood were lower than those in plasma, as shown in TABLE 4, because the 4-min reaction (hydrolysis) time applied in the biosensor in whole blood is longer than the 2-min reaction (hydrolysis) time selected in the biosensor in plasma. These results indicate that the sensitivity of the biosensor with ODI-CL detection is dependent on the reaction time for the hydrolysis reaction between protease enzyme (e.g., factors IIa, Xa) and substrate conjugated with AMC. Thus, it is expected that the LOD of the biosensor with ODI-CL detection may vary depending on the incubation time for the hydrolysis reaction between a fluorogenic substrate and the coagulation factor IIa (or Xa) in plasma or whole blood.

TABLE 4

| | | Sample | Analytical | Dynamic | | |
|---|---|---|---|---|---|---|
| Method | Factor | type | time (min) | range (nM) | LOD (pM) | References |
| Self-powered | IIa | Plasma | 30 | 1-100 | 410 | (Jung et al. |
| triboelectric aptasensor | | | | | | 2016)* |
| Bio-dots/AuNPs | IIa | Serum | 60 | 0-35 | 1,050 | (Kuang et al. |
| nanosensor with FRET | | | | | | 2016)** |
| detection | | | | | | |
| Aptasensor with | IIa | Serum/ | 150 | 0-0.05 | 0.01 | (Hao and Zhao |
| fluorogenic substrate | | plasma | | | | 2016)*** |
| Cyclic peptide coated | IIa | plasma | 180 | 0.005-0.2 | 5 | (Zhao and Gao |
| magnetic bead with | | | | | | 2015)**** |
| fluorogenic substrate | | | | | | |
| Colorimetric assay | IIa | Plasma | 90 | 0.001-0.1 | 0.2 | (Chen et al. |
| with chromogenic | | | | | | 2010)***** |
| substrate | | | | | | |
| Fluorometric Assay kit | Xa | Plasma | 30-60 | 0-4.4 | 444 | (MyBiosource) |
| | | | | | | ****** |
| Biosensor with ODI- | IIa | Plasma | 2 | 0.3-27.2 | 104 | This research |
| CL detection | | | | | | |
| Biosensor with ODI- | IIa | Whole | 4 | 0.21-10 | 66 | This research |
| CL detection | | blood | | | | |
| Biosensor with ODI- | Xa | Plasma | 2 | 0.25-20 | 44 | This research |
| CL detection | | | | | | |
| Biosensor with ODI- | Xa | Whole | 4 | 0.06-5 | 18 | This research |
| CL detection | | blood | | | | |

*Jung, Y.K., Kim, K.N., Baik, J.M., Kim, B.-S., 2016 Self-powered triboelectric aptasensor for label-free highly specific thrombin detection. Nano Energy 30, 77-83.
**Kuang, L., Cao, S.P., Zhang, L., Li, Q.H., Liu, Z.C., Liang, R.P., Qiu, J.D., 2016. A novel nanosensor composed of aptamer bio-dots and gold nanoparticles for determination of thrombin with multiple signals. Biosens Bioelectron 85, 798-806.
***Hao, L.H., Zhao, Q., 2016. Microplate based assay for thrombin detection using an RNA aptamer as affinity ligand and cleavage of a chromogenic or a fluorogenic peptide substrate. Microchim Acta 183(6), 1891-1898.
**** Zhao, Q., Gao, J., 2015. Sensitive and selective detection of thrombin by using a cyclic peptide as affinity ligand. Biosens Bioelectron 63, 21-25.
***** Chen, C.K., Huang, C.C., Chang, H.T., 2010. Label-free colorimetric detection of picomolar thrombin in blood plasma using a gold nanoparticle-based assay. Biosens Bioelectron 25(8), 1922-1927.
******(https://www.mybiosource.com/prods/Assay-Kit/Factor-Xaidatasheet.php? products_id=841634)

TABLE 4 shows that the sensitivity of a biosensor with ODI-CL detection, capable of quantifying IIa and Xa in plasma and whole blood, is as low as other methods operated with 10˜100 fold diluted human samples such as serum and plasma.

The fluorogenic substrate for the coagulation factors IIa and Xa having a fluorescent dye (AMC) have good specificity and selectivity. FIGS. 12A and 12B show that the fluorogenic substrate of the present invention does not react with other main proteins (e.g., Glucose, Hemoglobin, HSA, IgG) existing in a whole blood. The relative CL intensity of biosensor in the absence and presence of main proteins may be enhanced with the extension of the incubation time because a whole blood (e.g., 10%) contains trace levels of IIa and Xa.

Also, FIGS. 6C and 6D are related to experiments that test whether the fluorogenic substrates for the coagulation factors IIa and Xa conjugated with AMC can specifically and selectively react with active IIa or Xa in the presence of anticoagulants. FIGS. 6C and 6D show that the fluorogenic substrates for the coagulation factors IIa and Xa conjugated with AMC applied in the biosensor have good specificity and selectivity. IIa substrate conjugated with AMC can specifically interact with active IIa not bound with IIa anticoagulant (e.g., Dabigatran) while Xa fluorogenic substrate can selectively react with active Xa not bound with Xa anticoagulant (e.g., Rivaroxaban). Thus, the biosensor confirmed that trace levels of active IL are present in patient whole blood with Dabigatran as shown in FIG. 6C. The relative CL intensity measured after the reaction between Xa and Xa substrate conjugated with AMC in patient whole blood with Dabigatran was strong because Xa doesn't bind with Dabigatran (FIG. 6C). As shown in FIG. 6D, the biosensor confirmed that the concentration of active Xa in patient whole blood with Rivaroxaban is very low. Also, FIG. 6D shows that the relative CL intensity measured after the reaction of IIa and IIa substrate conjugated with AMC was strong in patient whole blood in the presence of Xa anticoagulant. In conclusion, the results shown in FIGS. 6C and 6D indicate that the biosensor operated with the substrates conjugated with AMC can be applied to prevent bleeding, thrombosis, and stroke with excellent selectivity and specificity.

TABLE 5 shows that the accuracy, precision, and recovery of the biosensor for whole blood are as good as those for human plasma.

TABLE 5
(Accuracy, precision, and recovery for the all-in-one
Biosensor with ODI-CL detection for the quantization of
IIa and Xa in whole blood (N = 5))

| | Sample 1 | Sample 2 | Expected | Measured | Recovery |
|---|---|---|---|---|---|
| Factor | (nM) | (nM) | (nM) | (nM) | (%) |
| IIₐ | 0.5 | 1.5 | 1.0 | 0.95 ± 0.05 | 95.9 |
| | 1.5 | 3.5 | 2.5 | 2.66 ± 0.18 | 106.8 |
| Xₐ | 0.8 | 2.0 | 1.4 | 1.35 ± 0.09 | 96.4 |
| | 1.4 | 4.2 | 2.8 | 2.63 ± 0.20 | 93.9 |

Accordingly, a biosensor with ODI-CL detection according to exemplary embodiments of the present invention rapidly quantify the coagulation factors IIa and Xa in whole blood with acceptable reproducibility as compared to conventional biosensors.

Additionally, TABLE 6 shows that the concentrations of IIa and Xa in whole blood quantified using the biosensor with ODI-CL detection are the same as those determined using the conventional method with fluorescence detection within the statistically acceptable error range.

TABLE 6
(Quantification of IIa and Xa in whole
blood using the biosensor with ODI-CL detection and
conventional method with fluorescence detection (N = 3))

| | Biosensor with | | Conventional method with | |
|---|---|---|---|---|
| | ODI-CL detection | | fluorescence detection | |
| | (4-min incubation) | | (30-min incubation) | |
| | IIa | Xa | IIa | Xa |
| Sample 1 | 0.98 (±0.05) | 0.63 (±0.04) | 0.95 (±0.06) | 0.67 (±0.05) |
| Sample 2 | 1.60 (±0.09) | 1.29 (±0.06) | 1.54 (±0.11) | 1.20 (±0.09) |
| Sample 3 | 2.85 (±0.12) | 2.22 (±0.11) | 2.99 (±0.16) | 2.03 (±0.14) |

A biosensor and a method of using a biosensor as described above may be provided in the form of a kit. In one embodiment of the present invention, the kit includes the above-described biosensor and a container. The kit may further include a buffer and an ODI-CL reagent (e.g., ODI and H₂O₂).

Accordingly, the present invention provides a cost-effective biosensor with ODI-CL detection which can be applied as a new device for rapid coagulation testing. The fluorescent dye (Luminophore) can be formed from the rapid reaction between coagulation factors (e.g., Xa) and a specific fluorogenic substrate. The intensity of light emitted with the addition of ODI-CL reagents (e.g., ODI, H₂O₂) in the solution was proportionally enhanced with the increase of the coagulation factor concentration in blood sample (e.g., plasma, whole blood). It is expected that the wide dynamic range of the biosensor with ODI-CL detection can diagnose and monitor bleeding and clotting in patients with statistically acceptable accuracy, precision, and reproducibility. In addition, the analytical procedure of the biosensor with ODI-CL detection is rapid and simple because sample pretreatment, time-consuming multiple incubations and washings aren't necessary. In conclusion, the concepts and principle of the biosensor with ODI-CL detection of the present invention can be widely applied for the early diagnosis and rapid monitoring of human diseases such as cancer, cardiac ailments, and infectious diseases (e.g., HIV, SARs, Zika virus).

### EXAMPLES

The experiments described in this specification were conducted with the following materials and procedures.

Chemicals and Materials

Thrombin from human plasma (coagulation factor IIa, 100 UN) and fluorogenic substrate of thrombin (Benzoyl-Phe-Val-Arg-AMC, HCl, 25 mg) were purchased from Sigma-Aldrich. Factor Xa (human) native protein was purchased from Invitrogen. Fluorogenic substrate of factor Xa (CH₃SO₂-D-CHA-Gly-Arg-AMC, AcOH) was purchased from Cryopep. AMC, as a fluorescent dye (fluorophore), is 7-Amino-4-methylcoumarin. Normal plasma lyophilized with pooled human dornors (1 g) was purchased from LEE Biosolution. Bis (2,4,6-trichlorophenyl) oxalate (TCPO) and 4-methylimidazole (4 MImH) were purchased from TCI America. 3 and 30% H₂O₂ were purchased from VWR. Deionized H₂O (HPLC grade), Ethyl acetate, Isopropyl alcohol, and high concentration of PBS (pH 7.4, 20×), TBS (pH 7.4×10), PBST and TBST were purchased from EMD. 8-well EIA/RIA strip-well plate was purchased from Costar. Human plasma and whole blood were provided by Meritus Medical Center, IIagerstown, Md., USA.

Confirmation of the Chemical Reaction Between Coagulation Factor IIa or Xa and a Specific Fluorogenic Substrate Using ODI-CL Detection

Experiment 1: Background of Fluorogenic Substrate Only in the Absence of Factor IIa or Xa in ODI-CL Reaction (FIG. 2A)

Each fluorogenic substrate (5 mg/ml) was dissolved in DMSO as a stock solution. The stock solution was stored in a freezer (−80° C.). The working solution of fluorogenic substrate (5 μg/ml) diluted in PBS (pH 7,4) was prepared before conducting the experiment. Each working solution (10 μl) was injected into a borosilicate test tube (12 mm×75 mm). The tube was inserted into the detection area of the luminometer (Lumat LB 9507, Berthold, Inc) with two syringe pumps. 100 mM H₂O₂ (25 μl) dissolved in isopropyl alcohol was dispensed through the first syringe pump of the luminometer. With the addition of ODI (25 μl) using the second syringe pump, we investigated whether each fluorogenic substrate can emit light in the absence of factor IIa or Xa. With this procedure, we were able to determine the background of fluorogenic substrate in the absence of factor IIa and Xa in ODI-CL reaction.

Experiment 2: CL Emission of AMC Formed from the Reaction of Fluorogenic Substrate and Coagulation Factor (FIG. 2A)

Each coagulation factor (IIa or Xa, 5 nM) was prepared in 10-fold diluted plasma with deionized H₂O. Each fluorogenic substrate (5 μg/ml) was prepared in PBS. The mixture of factor IIa (50 μl) and fluorogenic substrate (50 μl) of factor IIa in a strip-well was incubated for 2 minutes under ambient condition. Also, the mixture of factor Xa (50 μl) and flurogenic substrate (50 μl) of factor Xa in a strip-well was also incubated for 2 minutes under ambient condition. After the incubation, each mixture (10 μl) was inserted into a borosilicate test tube. H₂O₂ (25 μl) and ODI (25 μl) were consecutively dispensed through two syringe pumps of the luminometer to measure relative CL intensity of light emitted in the tube.

Experiment 3: Sensitivity of Fluorescence and ODI-CL for the Quantification of Coagulation Factor (TABLE 2)

12 standards (0-10 nM) of factor Xa in 10% human plasma were prepared. Fluorogenic substrate (5 μg/ml) of factor Xa was prepared in PBS. Each standard solution (50 μl) was mixed with fluorogenic substrate (50 μl) in a strip-well. The mixture was incubated for 2 minutes under ambient condition. After the incubation, relative CL intensity of each sample was measured using the luminometer operated with the same method described in Experiments 1 and 2. In order to measure fluorescence intensity of each sample, the mixture in the strip-well was incubated for 30 min under ambient condition. After the incubation, the strength of fluorescence emitted in the strip-well was measured with a microplate reader (Infinite M 1000 of Tecan, Inc.). Finally, the sensitivity of ODI-CL detection for the quantification of coagulation factor was compared with that of fluorescence detection.

Experiment 4: Quantification of Coagulation Factors in Human Plasma Using the Biosensor with ODI-CL Detection (TABLE 3)

Standards of factor IIa and Xa were prepared with 10% plasma diluted with deionized H₂O. Unknown samples were prepared with 100% plasma. Then, each sample was 10-fold diluted in deionized H₂O. Each standard or sample (50 μl) was dispensed into a strip-well containing fluorogenic substrate. The mixture in the strip-well was incubated for 2 minutes under ambient condition. The fluorogenic substrate of factor IIa (5 μg/ml) was prepared in TBST. Also, the fluorogenic substrate of factor Xa (5 μg/ml) was prepared in PBS. After the incubation, light emitted from each mixture with the addition of ODI CL reagents was measured for 2 sec using the luminometer.

Experiment 5: Quantification of Coagulation Factors in Human Whole Blood Using the Biosensor with ODI-CL Detection (TABLE 5)

Standards of factor IIa and Xa were prepared with 10% whole blood diluted with deionized H₂O. Unknown whole blood samples were 10-fold diluted in deionized H₂O. Each standard or sample (50 μl) was dispensed into a strip-well containing fluorogenic substrate. The mixture in the strip-well was incubated for 4 minutes under ambient condition. The fluorogenic substrate of factor IIa (25 μg/ml) was prepared in TBST. Also, the fluorogenic substrate of factor Xa (25 μg/ml) was prepared in PBS. After the 4-min incubation, light emitted from each mixture with the addition of ODI-CL reagents was measured for 2 sec using the luminometer.

Experiment 6: Correlation Between Biosensor with ODI-CL Detection and Conventional Method with Fluorescence Detection for the Quantification of IIa and Xa in 10-Fold Diluted Plasma and Whole Blood (TABLE 4)

In order to confirm the correlation between the biosensor with ODI-CL detection and the conventional method with fluorescence detection, the concentrations of IIa and Xa in 10-fold diluted plasma or whole blood (e.g., standards, samples) were determined with a microplate reader with fluorescence detection (Infinite M 1000, Tecan, Inc). The concentrations of fluorogenic substrates of IIa and Xa for the quantification of IIa and Xa using the conventional method were the same as those using the biosensor with ODI-CL detection described in Experiments 4 and 5. Each standard or sample (50 μl) was mixed with fluorogenic substrate (50 μl) in a black well. The black well-plate (96 well, Greiner Bio-One) containing various mixtures, was inserted into the microplate reader with fluorescence detection and incubated for 30 min at room temperature. After the incubation, the relative intensity of fluorescence emitted from each well was measured at 440 nm emission wavelength (excitation wavelength: 342 nm). After determining the concentrations of samples in plasma and whole blood using the conventional method, they were compared with those that used the biosensor with ODI-CL detection to confirm the correlation between the new and conventional methods.

Analysis of Experimental Data

All experimental results observed in this specification were analyzed using the statistical tools of Microsoft Excel and SigmaPlot 12.5 (Systat software, Inc.).

It is to be understood that the above-described biosensor and method are merely illustrative embodiments of the principles of this disclosure, and that other compositions and methods for using them may be devised by one of ordinary skill in the art, without departing from the spirit and scope of the invention. It is also to be understood that the disclosure is directed to embodiments both comprising and consisting of the disclosed parts.

What is claimed is:

1. A biosensor for detecting a coagulation factor in a blood sample comprising:
a fluorogenic substrate for the coagulation factor, wherein the fluorogenic substrate includes a fluorescent dye; and
a quencher conjugated with the fluorogenic substrate.

2. The biosensor of claim 1, wherein the coagulation factor is coagulation factor IIa or Xa.

3. The biosensor of claim 1, wherein the blood sample is plasma or whole blood.

4. The biosensor of claim 1, wherein the blood sample is 1 to 1,000-fold diluted plasma or whole blood.

5. The biosensor of claim 1, wherein the fluorescent dye is at least one selected from the group consisting of 2-aminobenzoyl (Abz), N-methyl-anthraniloyl (N-Me-Abz). 5-(dimethylamino)naphthalene-1-sulfonyl (Dansyl), 5-[(2-aminoethyl)amino]-naphthalene-1-sulfonic acid (EDANS), 7-dimethylaminocoumarin-4-acetate (DMACA), 7-amino-4-methylcoumarin (AMC), (7-methoxycoumarin-4-yl)acetyl (MCA), rhodamine, rhodamine 101, rhodamine 110 and resorufin.

6. The biosensor of claim 1, wherein the fluorescent dye emits light when:
the fluorescent dye dissociates from the fluorogenic substrate by a hydrolysis reaction between the coagulation factor and the fluorogenic substrate; and
the fluorescent dye interacts with high-energy intermediate formed from 1, 1′-oxalyldiimidazole chemiluminescence (ODI-CL) reagent.

7. The biosensor of claim 1, wherein the 1,1′-oxalyldiimidazole chemiluminescence (ODI-CL) reagent comprises an ODI and H₂O₂.

8. The biosensor of claim 1, wherein the quencher is at least one selected from the group consisting of 2,4-Dinitrophenyl (DNP), N-(2,4-Dinitrophenyl)ethylenediamine (EDDnp), 4-Nitro-phenylalanine, 3-Nitro-tyrosine, para-Nitroaniline (pNa), 4-(4-Dimethylaminophenylazo)benzoyl (DABCYL) and 7-Nitro-benzo[2,1,3]oxadiazol-4-yl (NBD).

9. A method of monitoring a coagulation factor in a blood sample, the method comprising:
mixing and reacting the biosensor of claim 1 with a blood sample including a coagulation factor in a buffer;
adding a 1,1′-oxalyldiimidazole chemiluminescence (ODI-CL) reagent to the reacted mixture; and
measuring CL intensity.

10. The method of claim 9, wherein the reaction time between the blood sample and the fluorogenic substrate in the biosensor at room temperature (21±2° C.) or 37° C. is 10 seconds to 120 minutes.

11. The method of claim 9, wherein measuring CL intensity is performed for 1 to 10 seconds after adding the ODI-CL reagent.

12. The method of claim 9, wherein the coagulation factor is coagulation factor IIa or Xa.

13. The method of claim 9, wherein the blood sample is plasma or whole blood

14. The method of claim 9, wherein the buffer is selected from the groups consisting of PBST, PBS, TBST and TBS.

15. A method of quantifying a coagulation factor in a blood sample, the method comprising:
mixing and reacting the biosensor of claim 1 with a blood sample including a coagulation factor in a buffer;
adding 1,1′-oxalyldiimidazole chemiluminescence (ODI-CL) reagent to the reacted mixture;
measuring CL intensity; and
comparing the intensity of CL with a standard intensity.

16. A kit for quantifying a coagulation factor in a blood sample, the kit comprising:
the biosensor of claim 1; and
a container.

17. The kit of claim 16 further comprising:
a buffer; and
1,1′-oxalyldiimidazole chemiluminescence (ODI-CL) reagent.
